(19) United States
(12) Patent Application Publication
(10) Pub. No.: US 2025/0228988 A1
(43) Pub. Date: Jul. 17, 2025

(54) FLUID STERILIZATION DEVICE

(71) Applicant: TOYODA GOSEI CO., LTD., Kiyosu-shi (JP)

(72) Inventors: Aya KAWAOKA, Kiyosu-shi (JP); Yusuke TOYODA, Kiyosu-shi (JP); Yoshiki SAITO, Kiyosu-shi (JP)

(21) Appl. No.: 18/990,288

(22) Filed: Dec. 20, 2024

(30) Foreign Application Priority Data
Jan. 11, 2024 (JP) 2024-002220
Jan. 11, 2024 (JP) 2024-002221
Jan. 11, 2024 (JP) 2024-002222

(51) Int. Cl. A61L 2/10; A61L 9/20; C02F 1/32

(52) U.S. Cl. CPC A61L 2/10; A61L 9/20; C02F 1/325; A61L 2202/11; A61L 2202/121; A61L 2202/122; A61L 2209/12; C02F 2201/3222; C02F 2303/04

(57) ABSTRACT

The fluid sterilization device includes a sterilization chamber body having a sterilization chamber whose wall surface is formed in a concave spherical shape, and a light source unit that blocks a light source aperture and is configured to emit ultraviolet light from the light source aperture into the sterilization chamber. The fluid sterilization device further includes a housing that is disposed so as to cover an outer surface of the sterilization chamber body, and has an outside region facing the outer surface of the sterilization chamber body and an outer surface of the light source unit, the outside region being configured to flow the fluid fed from the housing inlet to the chamber inlet. The light source unit includes a light emitting element configured to emit the ultraviolet light.

### CROSS-REFERENCE

This application claims priority to Japanese patent application Nos. JP 2024-002220 filed on Jan. 11, 2024, JP 2024-002221 filed on Jan. 11, 2024, and JP 2024-002222 filed on Jan. 11, 2024, the contents of which are fully incorporated herein by reference.

### BACKGROUND OF THE INVENTION

### Technical Field

The present disclosure relates to a fluid sterilization device.

### Background Art

Sterilization devices are known to kill and inactivate bacteria and viruses in running water by irradiating ultraviolet light. Mercury lamps are widely used as light sources. Mercury lamps are highly toxic because they use mercury, and they have a large environmental impact. In addition, the use of mercury lamps also causes the sterilization equipment to be large in size. Therefore, mercury lamps are being replaced by ultraviolet LEDs.

JP-A-2020-530384 discloses a fluid sterilization device that can sterilize fluid by irradiating the fluid with ultraviolet light. The sterilization device includes a housing for fluid flow. The housing has a sterilization chamber body having a sterilization chamber for irradiating fluid with ultraviolet light and disposed to cover the outer surface of the sterilization chamber body. A gap (outside region) is provided between the sterilization chamber body and the housing, and the fluid flowing into the housing flows into the sterilization chamber body through the outside region.

### SUMMARY OF THE INVENTION

However, in the fluid sterilization device disclosed in JP-A-2020-530384, there was room for improving the sterilization performance by improving the performance of the light emitting element. In addition, in the fluid sterilization device disclosed in JP-A-2020-530384, there was a possibility that the sterilization performance would vary, and it was desirable to stabilize the sterilization performance.

The present disclosure is made in view of such background, and intends to provide a fluid sterilization device with improved sterilization performance. The present disclosure also intends to provide a fluid sterilization device with improved stability of sterilization performance.

One aspect of the present disclosure is a fluid sterilization device comprising:
a sterilization chamber body including
   a sterilization chamber for a fluid, a wall surface of which has a concave spherical shape,
   a light source aperture,
   a chamber inlet for inflowing the fluid into the sterilization chamber, and
   a chamber outlet for outflowing the fluid from the sterilization chamber, the light source aperture, the chamber inlet, and the chamber outlet being formed so as to be open to the sterilization chamber;
a light source unit that blocks the light source aperture and is configured to emit ultraviolet light from the light source aperture into the sterilization chamber; and
a housing that is disposed so as to cover an outer surface of the sterilization chamber body, has a housing inlet for feeding the fluid formed thereon, and has an outside region, which faces the outer surface of the sterilization chamber body and an outer surface of the light source unit, formed therein, the outside region being configured to flow the fluid fed from the housing inlet to the chamber inlet, wherein
the light source unit includes a light emitting element configured to emit the ultraviolet light.

The above mentioned aspect makes it possible to provide a fluid sterilization device having enhanced sterilization performance.

The abovementioned aspect makes it possible also to provide a fluid sterilization device in which stability of the sterilization performance is enhanced.

### BRIEF DESCRIPTION OF THE DRAWINGS

FIG. 1 is a cross-sectional view of a fluid sterilization device in a first embodiment;

FIG. 2 is a schematic view illustrating the flow of fluid in the fluid sterilization device;

FIG. 3 shows the configuration of the sterilization chamber body. FIG. 3(a) is an exploded view of the sterilization chamber body 10, and FIG. 3(b) shows the sterilization chamber body 10 in the assembled state;

FIG. 4 shows the positions of a light source aperture, a chamber inlet, and a chamber outlet when viewing the outer side of the sterilization chamber body from the central axis direction of the light source aperture;

FIG. 5 is a cross-sectional view showing the configuration of a light source unit;

FIG. 6 shows the position of a housing inlet and the light source unit when viewing the outside of the housing from the central axis direction of the housing inlet;

FIG. 7 is an enlarged cross-sectional view of the connection between the chamber outlet and a housing outlet;

FIG. 8 shows the results of water flow simulation;

FIG. 9 shows an example of the orientation characteristics of the light emitting element;

FIG. 10 shows an example of the emission spectrum of the light emitting element; and

FIG. 11 shows an example of the electrode pattern of the light emitting element.

### DETAILED DESCRIPTION OF PREFERRED EMBODIMENTS

A fluid sterilization device includes: a sterilization chamber body including a sterilization chamber for a fluid, a wall surface of which has a concave spherical shape, a light source aperture, a chamber inlet for inflowing the fluid into the sterilization chamber, and a chamber outlet for outflowing the fluid from the sterilization chamber, the light source aperture, the chamber inlet, and the chamber outlet being formed so as to be open to the sterilization chamber; a light source unit that blocks the light source aperture and is configured to emit ultraviolet light from the light source aperture into the sterilization chamber; and a housing that is disposed so as to cover an outer surface of the sterilization chamber body, has a housing inlet for feeding the fluid formed thereon, and has an outside region, which faces the outer surface of the sterilization chamber body and an outer surface of the light source unit, formed therein, the outside region being configured to flow the fluid fed from the housing inlet to the chamber inlet. The light source unit includes a light emitting element configured to emit the ultraviolet light.

In the fluid sterilization device, the light emitting element may be configured to have an emission efficiency of 2.4% or more at a current value within a range of 300 mA to 400 mA. The sterilization performance can be enhanced.

In the fluid sterilization device, the light emitting-element may be configured to have an emission efficiency of 2.5% or more at a current value of 350 mA. The sterilization performance can be further enhanced.

In the fluid sterilization device the light emitting element may be configured such that in an emission spectrum, a difference between a maximum half width value and a minimum half width value is 2 nm or less at a current value within a range of 200 mA to 500 mA. The stability of sterilization performance can be enhanced.

In the fluid sterilization device, the light emitting element may be configured such that in the emission spectrum, the difference between the maximum half width value and the minimum half width value is 1 nm or less at the current value within the range of 200 mA to 500 mA. The stability of sterilization performance can be further enhanced.

In the fluid sterilization device, the light emitting element may be configured such that an emission efficiency at one of current values within a range of 10 mA to 50 mA takes a maximum value among emission efficiencies at applied current values, and such that the emission efficiencies at current values in an entire range of 200 mA to 500 mA are 75% or more relative to a maximum value of the emission efficiency at a current value within a range of 200 mA to 500 mA. The stability of sterilization performance can be enhanced.

In the fluid sterilization device, the light emitting element may be configured such that the emission efficiencies at current values in the entire range of 200 mA to 500 mA fall within a range of 65% to 85% relative to the maximum value of the emission efficiency at the applied current values. More stable sterilization performance can be obtained.

In the fluid sterilization device, the light source unit may include a light-emitting surface that is located corresponding to the light source aperture and is configured to emit ultraviolet light, and an external back surface that forms part of the outer surface of the light source unit, the external back surface being an opposite side surface of the light-emitting surface with which the fluid flowing through the outside region comes into contact. The housing inlet may be arranged such that an opening of the housing inlet faces the external back surface of the light source unit. Thus, the light source unit can be efficiently cooled, and the sterilization performance can be improved.

### First Embodiment

### 1. Basic Configuration of Fluid Sterilization Device 1

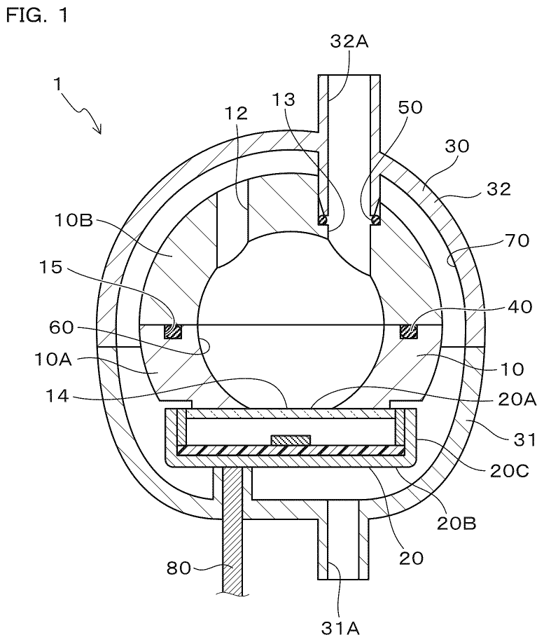

The basic configuration of the fluid sterilization device 1 is described with reference to FIG. 1. As shown in FIG. 1, the fluid sterilization device 1 includes mainly a sterilization chamber body 10 having a sterilization chamber 60, a light source unit 20 that emits ultraviolet light into the sterilization chamber 60, and a housing 30 that includes the sterilization chamber body 10 and the light source unit 20.

A space is formed between the sterilization chamber body 10 and the housing 30. Since that space is disposed outside of the sterilization chamber body 10, the space is referred to as an outside region 70. Furthermore, the fluid sterilization device 1 has a first seal member 40 and a second seal member 50. The first seal member 40 and the second seal member 50 will be described later.

The fluid sterilization device 1 is a device that flows a fluid from the outside into the sterilization chamber 60 through the outside region 70 and irradiates the fluid in the sterilization chamber 60 with ultraviolet light from the light source unit 20 to sterilize the fluid. The fluid to be sterilized may be a gas or a liquid, or a mixture of gas and liquid or a mixture of gas and powdered solid as long as it is flowable. In the case of a liquid, the fluid to be sterilized is, for example, water, oil, alcohol, or a dissolved solution using these as solvents.

The sterilization chamber body 10 includes a sterilization chamber 60 inside. The sterilization chamber 60 is a space where the flowing fluid is irradiated with the ultraviolet light emitted from the light source unit 20. The wall surface of the sterilization chamber 60 is formed in a concave spherical shape. Thus, ultraviolet light can be efficiently reflected by the concave spherical shape, and the illuminance of the ultraviolet light in the sterilization chamber 60 can be increased, thereby improving the sterilization efficiency of the fluid.

The sterilization chamber body 10 is made of a material with a high ultraviolet reflectance. For example, the sterilization chamber body 10 is entirely made of PTFE (polytetrafluoroethylene). The use of PTFE enhances ultraviolet light reflectance, thereby improving the sterilization efficiency. Any material other than PTFE may be used as long as it has a high reflectance to the ultraviolet light from the light source unit 20. In particular, the material of the sterilization chamber body 10 may be a material with a reflectance of 80% or higher, preferably 90% or higher, and more preferably 95% or higher to the ultraviolet light from the light source unit 20. The sterilization chamber body 10 may be formed so that only the surface layer having a concave spherical surface is made of a material with a reflectance of 80% or more to ultraviolet light, for example, PTFE or aluminum.

The sterilization chamber body 10 has a light source aperture 14 that opens into the sterilization chamber 60. The light source aperture 14 is an opening for the ultraviolet light emitted from the light source unit 20 to enter the sterilization chamber 60.

The sterilization chamber body 10 further has a chamber inlet 12 that opens into the sterilization chamber 60. The chamber inlet 12 provides a conduit between the sterilization chamber 60 and the outside region 70. The chamber inlet 12 is an inlet for fluid to flow from the outside region 70 into the sterilization chamber 60.

Additionally, the sterilization chamber body 10 has a chamber outlet 13 that opens into the sterilization chamber 60. The chamber outlet 13 provides a conduit between the sterilization chamber 60 and the outside. The chamber outlet 13 is an outlet for fluid to flow out from the sterilization chamber 60 to the outside.

The light source unit 20 is disposed so as to block the light source aperture 14. The light source unit 20 is configured to emit ultraviolet light from the light source aperture 14 into the sterilization chamber 60. The portion exposed to the light source aperture 14 of the light source unit 20, i.e., the light-emitting surface 20A of the light source unit 20 that emits the ultraviolet light, forms part of the wall surface of the sterilization chamber 60. Therefore, the fluid in the sterilization chamber 60 comes into contact with the light-emitting surface 20A of the light source unit 20. Thus, the light source unit 20 is cooled by the fluid in the sterilization chamber 60. As a result, the emission efficiency of the light source unit 20 can be increased.

The housing 30 is formed to include the sterilization chamber body 10 and the light source unit 20. In other words, the housing 30 is disposed so as to cover the sterilization chamber body 10 and the light source unit 20. In detail, the inner surface of the housing 30 faces the outer surface of the sterilization chamber body 10. The inner surface of the housing 30 faces the outer surface of the light source unit 20. In other words, the inner surface of the housing 30 faces an external back surface 20B and an outer peripheral surface 20C that form the outer surface of the light source unit 20.

The gap between the inner surface of the housing 30 and the outer surface of the sterilization chamber body 10 as well as the gap between the inner surface of the housing 30 and the outer surface of the light source unit 20 form the outside region 70. A part of the outside region 70 is the area facing the inner surface of the housing 30 and facing the outer surface of the sterilization chamber body 10. The other part of the outside region 70 is the area facing the inner surface of the housing 30 and facing the outer surface of the light source unit 20.

The housing inlet 31A for feeding the fluid is formed in the housing 30. The housing inlet 31A leads to the outside region 70. That is, the fluid fed from the housing inlet 31A passes through the outside region 70 and flows into the sterilization chamber 60 from the chamber inlet 12.

The housing outlet 32A for discharging the fluid is further formed in the housing 30. The housing outlet 32A leads to the chamber outlet 13 of the sterilization chamber body 10. Thus, the fluid sterilized in the sterilization chamber 60 is discharged to the outside from the housing outlet 32A through the chamber outlet 13.

### 2. Fluid Flow Path in Fluid Sterilization Device 1

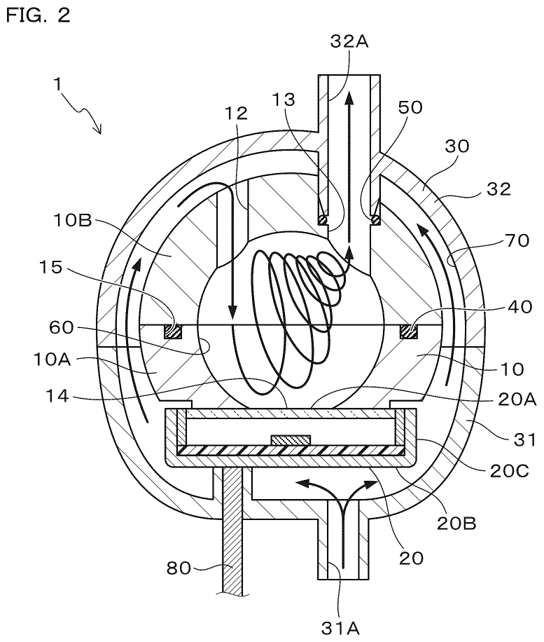

The fluid flow path in the fluid sterilization device 1 is described with reference to FIG. 2. As shown in FIG. 2, fluid is fed from the housing inlet 31A to the outside region 70.

Firstly, the fluid fed from the housing inlet 31A flows into the gap between the inner surface of the housing 30 and the external back surface 20B of the light source unit 20 in the outside region 70. At this time, the fluid contacts the external back surface 20B of the light source unit 20 to cool the light source unit 20. The fluid then flows into the gap between the inner surface of the housing 30 and the outer peripheral surface 20C of the light source unit 20 in the outside region 70. The fluid contacts the outer peripheral surface 20C of the light source unit 20 to cool the light source unit 20. The fluid then flows into the gap between the inner surface of the housing 30 and the outer surface of the sterilization chamber body 10 in the outside region 70.

Thus, the fluid fed from the housing inlet 31A firstly contacts the external back surface 20B and the outer peripheral surface 20C, which are the outer surfaces of the light source unit 20. Therefore, the light source unit 20 is efficiently cooled by the fluid fed from the housing inlet 31A. As a result, the emission efficiency of the light source unit 20 can be increased.

The fluid in the outside region 70 then flows into the sterilization chamber 60 from the chamber inlet 12 of the sterilization chamber body 10. The fluid that flows into the sterilization chamber 60 proceeds toward the chamber outlet 13 while maintaining a spiral flow. The fluid flows from the chamber outlet 13 to the housing outlet 32A and is discharged to the outside.

Here, the fluid in the sterilization chamber 60 forms a spiral flow because the sterilization chamber 60 has a concave spherical shape wall surface. Furthermore, the chamber inlet 12 and outlet 13 are configured such that the direction of the central axis of the spiral flow generated along the concave spherical shape wall surface in the sterilization chamber 60 changes as the spiral flow proceeds from near the chamber inlet 12 to the chamber outlet 13.

As shown in FIG. 2, the direction of the central axis of the spiral flow in the sterilization chamber 60 changes from the chamber inlet 12 toward the chamber outlet 13. Due to the positional relationship of the chamber inlet 12 and the chamber outlet 13, the direction of the central axis of the spiral flow of the fluid flowing into the sterilization chamber 60 changes so as to approach the direction of the central axis L3 of the chamber outlet 13.

In this embodiment, since the direction of the central axis of the spiral flow generated immediately after flowing in from the chamber inlet 12, has an angle with respect to the direction of the central axis L3 of the chamber outlet 13, the direction of the central axis of the spiral flow changes. In particular, the angle between the direction of the central axis of the spiral flow and the direction of the central axis L3 of the chamber outlet 13 is large (close to 90°), and so the direction of the central axis of the spiral flow changes at a large angle. With this, stagnation of the fluid is prevented, enabling the fluid to flow well in the sterilization chamber 60. In particular, the fluid flow in the vicinity of the chamber outlet 13 can be made good.

In this way, in the sterilization chamber 60, the fluid flowing in from the chamber inlet 12 can be suppressed from immediately flowing toward the chamber outlet 13, and a spiral flow can be effectively generated. Since the spiral flow can be maintained in the entire sterilization chamber 60, the flow path of the fluid in the sterilization chamber 60 can be lengthened. As a result, a desired cumulative irradiation amount of the ultraviolet light from the light source unit 20 can be achieved, and the desired sterilization performance can be ensured. Furthermore, the pressure loss can be reduced, and the sterilization efficiency can be increased.

In addition, the pressure loss of the fluid in the sterilization chamber 60 can be reduced because the spiral flow can be maintained in the sterilization chamber 60. This also improves the sterilization efficiency.

### 3. Components of Sterilization Chamber Body 10

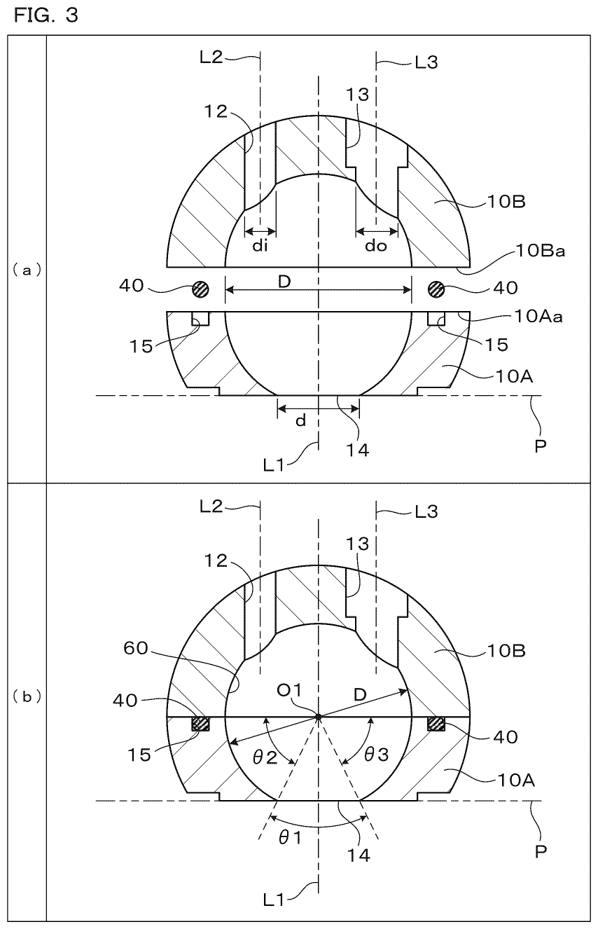

The components of the sterilization chamber body 10 are described with reference to FIGS. 3 (a) and (b). The sterilization chamber body 10 includes a plurality of body components divided into at least two parts. The sterilization chamber body 10 formed by two body components is given as an example.

The sterilization chamber body 10 is divided into two parts: a first body component member 10A and a second body component member 10B, as shown in FIG. 3.

The first body component member 10A and the second body component member 10B are formed so that the sterilization chamber 60 is divided into two hemispherical parts. In other words, the first body component member 10A is one of a plurality of body component members that form the sterilization chamber body 10, and has a wall surface formed in a semi-concave spherical shape. The second body component member 10B is one of a plurality of body component members that form the sterilization chamber body 10, and has a wall surface formed in a semi-concave spherical shape. The second body component member 10B is disposed facing the first body component member 10A.

Ring-shaped boundary surfaces 10Aa and 10Ba appear as division surfaces in the first body component member 10A and the second body component member 10B, respectively. The sterilization chamber 60 is formed inside the first body component member 10A and the second body component member 10B by abutting the ring-shaped boundary surface 10Aa and the ring-shaped boundary surface 10Ba.

The diameter of the concave spherical surface of the sterilization chamber body 10 is D. The diameter of the opening of the sterilization chamber 60 in the division surface between the first body component member 10A and the second body component member 10B corresponds to D. By dividing the sterilization chamber body 10 into two parts in this manner, the sterilization chamber body 10 can be easily fabricated.

A light source aperture 14 is formed in the first body component member 10A. The plane P including the light source aperture 14 is parallel to the boundary surfaces 10Aa and 10Ba. In other words, the central axis L1 of the light source aperture 14 is orthogonal to the boundary surfaces 10Aa and 10Ba. Accordingly, the light source aperture 14 is formed farthest from the boundary surfaces 10Aa and 10Ba in the first body component member 10A. The edge line of the light source aperture 14 is, for example, a circle. Thus, the light source aperture 14 is disposed in the same plane. The diameter of the edge line of the light source aperture 14 is d.

The ratio D/d of the diameter D of the sterilization chamber 60 and the diameter d of the light source aperture 14 is set, for example, in a range of 1.8 to 2.2. In this case, when the center point O1 of the sterilization chamber 60 is set as a center, the opening angle θ1 of the light source aperture 14 formed in the first body component member 10A is approximately 60°. On the same basis, the sum of the angles θ2 and θ3 of the portion for forming a concave spherical shape of the first body component member 10A is the remaining 120° (60° each). Thus, the fluid flowing in from the chamber inlet 12 certainly hits the concave spherical wall surface, thereby effectively generating a spiral flow. As a result, the pressure loss can be reduced, and the sterilization efficiency can be improved.

The second body component member 10B has the chamber inlet 12 and the chamber outlet 13. The central axis L2 of the chamber inlet 12 is offset from the center point O1 of the sterilization chamber 60. The central axis L2 of the chamber inlet 12 is parallel to the central axis L1 of the light source aperture 14. However, if the central axis L2 of the chamber inlet 12 is offset from the center point O1, the central axis L2 of the chamber inlet 12 and the central axis L1 of the light source aperture 14 may be intersected or twisted to each other.

The chamber inlet 12 has, for example, a cylindrical inner surface. The inner diameter of the chamber inlet 12 is di. Since the central axis L2 of the chamber inlet 12 is offset from the center point O1 of the sterilization chamber 60, the edge line of the opening on the sterilization chamber 60 side in the chamber inlet 12 has a shape approximate to an egg shape.

The chamber inlet 12 is disposed such that the opening on the sterilization chamber 60 side in the chamber inlet 12 faces the surface on the sterilization chamber 60 side of the plane P including the light source aperture 14. In FIG. 3(b), the opening on the sterilization chamber 60 side in the chamber inlet 12 faces downward, and the surface on the sterilization chamber 60 side of the plane including the light source aperture 14 faces upward.

The central axis L3 of the chamber outlet 13 is offset from the center point O1 of the sterilization chamber 60. The central axis L3 of the chamber outlet 13 is parallel to the central axis L2 of the chamber inlet 12. Therefore, the central axis L3 of the chamber outlet 13 is formed parallel to the central axis L1 of the light source aperture 14 as well. However, if the central axis L3 of the chamber outlet 13 is offset from the center point O1, the central axis L3 of the chamber outlet 13 and the central axis L2 of the chamber inlet 12 may be intersected or twisted to each other. The central axis L3 of the chamber outlet 13 and the central axis L1 of the light source aperture 14 also may be intersected or twisted each other.

The chamber outlet 13 has, for example, a cylindrical inner surface. The inner diameter of the chamber outlet 13 is do. Since the central axis L3 of the chamber outlet 13 is offset from the center point O1 of the sterilization chamber 60, the edge line of the opening on the sterilization chamber 60 side in the chamber outlet 13 has a shape approximate to an egg shape.

The chamber outlet 13 is disposed such that the opening on the sterilization chamber 60 side in the chamber outlet 13 faces the surface on the sterilization chamber 60 side of the plane P including the light source aperture 14. In FIG. 3(b), the opening on the sterilization chamber 60 side in the chamber outlet 13 faces downward, and the surface on the sterilization chamber 60 of the plane including the light source aperture 14 faces upward.

As mentioned above, the central axis L1 of the light source aperture 14 is orthogonal to the boundary surfaces 10Aa and 10Ba. In this case, the entire surface of boundary surfaces 10Aa and 10Ba is a surface having the greatest angle with respect to the central axis L1 of the light source aperture 14. Thus, ultraviolet light entering the boundary surfaces 10Aa and 10Ba can be reduced, thereby reducing the leakage of ultraviolet light through the boundary surfaces 10Aa and 10Ba.

### 4. Positional Relationship of Openings 12, 13, and 14 of Sterilization Chamber Body

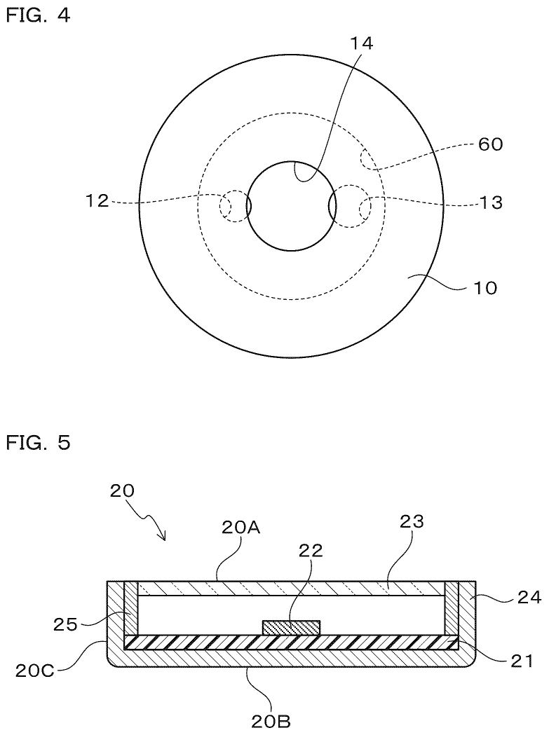

The positional relationship of the openings 12, 13, and 14 of the sterilization chamber body 10 is described with reference to FIG. 4. In detail, the positional relationship of the chamber inlet 12, the chamber outlet 13, and the light source aperture 14 is described.

As mentioned above, the central axes L1 of the light source aperture 14, L2 of the chamber inlet 12, and L3 of the chamber outlet 13 are parallel, for example. And the edge line of the light source aperture 14, the cross-sectional shape of the inner circumference of the chamber inlet 12, and the cross-sectional shape of the inner circumference of the chamber outlet 13 are circular, respectively.

The chamber inlet 12 is disposed such that the opening of the chamber inlet 12 faces the surface on the sterilization chamber 60 side of the plane P including the light source aperture 14. The chamber inlet 12 is disposed such that at least a part of the opening of the chamber inlet 12 does not overlap the light source aperture 14 when viewed from the direction of the central axis L1 of the light source aperture 14. In this embodiment, the chamber inlet 12 is disposed such that the other part of the opening of the chamber inlet 12 overlaps the light source aperture 14 when viewed from the direction of the central axis L1 of the light source aperture 14. In other words, the chamber inlet 12 is disposed such that only at least a part of the chamber inlet 12 is outside the light source aperture 14 when viewed from the direction shown in FIG. 4.

As mentioned above, the chamber inlet 12 is disposed so that at least a part of the opening of the chamber inlet 12 does not overlap the light source aperture 14 when viewed from the direction of the central axis L1 of the light source aperture 14. The central axis L1 of the light source aperture 14 is parallel to the central axis L2 of the chamber inlet 12. In other words, even when viewed from the direction of the central axis L2 of the chamber inlet 12, the chamber inlet 12 is disposed so that at least a part of the opening of the chamber inlet 12 does not overlap the light source aperture 14.

Therefore, all the fluid flowing in from the chamber inlet 12 does not proceed toward the light source aperture, and at least some of the fluid flowing in hits the concave spherical wall surface of the sterilization chamber 60. Thus, a spiral flow can be generated in the sterilization chamber by some of the fluid hitting the concave spherical wall surface of the sterilization chamber. By generating the spiral flow immediately after flowing in the sterilization chamber 60, the spiral flow can be maintained in the entire space of the concave spherical shape sterilization chamber 60. Thus, the desired cumulative irradiation amount can be achieved, and the desired sterilization performance can be ensured.

In addition to the above configuration, the chamber inlet 12 may be disposed so that the entire opening of the chamber inlet 12 does not overlap the light source aperture 14 when viewed from the direction of the central axis L1 of the light source aperture 14. That is, the chamber inlet 12 may be disposed so that the entire opening of the chamber inlet 12 does not overlap the light source aperture 14 when viewed from the direction of the central axis L2 of the chamber inlet 12. In this case, a spiral flow can be effectively generated in the sterilization chamber 60. By generating a stronger spiral flow immediately after flowing into the sterilization chamber 60, the spiral flow can be maintained in the entire space of the concave spherical shape sterilization chamber 60.

The chamber outlet 13 is disposed such that the opening of the chamber outlet 13 faces the side on the sterilization chamber 60 of the plane P including the light source aperture 14. The chamber outlet 13 is disposed such that at least a part of the opening of the chamber outlet 13 does not overlap the light source aperture 14 when viewed from the direction of the central axis L1 of the light source aperture 14. In this embodiment, the chamber outlet 13 is disposed so that the other part of the opening of the chamber outlet 13 overlaps the light source aperture 14 when viewed from the direction of the central axis L1 of the light source aperture 14. In other words, the chamber outlet 13 is also disposed such that only at least a part of the chamber outlet 13 is outside the light source aperture 14 when viewed from the direction shown in FIG. 4.

As mentioned above, the chamber outlet 13 is disposed so that at least part of the opening of the chamber outlet 13 does not overlap the light source aperture 14 when viewed from the direction of the central axis L1 of the light source aperture 14. The central axis L1 of the light source aperture 14 and the central axis L3 of the chamber outlet 13 are parallel. In other words, even when viewed from the direction of the central axis L3 of the chamber outlet 13, the chamber outlet 13 is disposed so that at least a part of the opening of the chamber outlet 13 does not overlap the light source aperture 14.

Thus, at least some of the fluid flowing toward the chamber outlet 13 flows toward the chamber outlet 13 while hitting the concave spherical wall surface of the sterilization chamber 60. The fluid flowing toward the chamber outlet 13 can be maintained in a spiral flow. As a result, the fluid flowing from chamber inlet 12 to the chamber outlet 13 can be entirely maintained in a spiral flow. Since the spiral flow can be maintained in the sterilization chamber 60, the pressure loss of the fluid in the sterilization chamber 60 can be reduced, and the sterilization efficiency can be increased. Therefore, the desired cumulative irradiation amount can be achieved, and the desired sterilization performance can be ensured.

In addition to the above configuration, the chamber outlet 13 may be disposed so that the entire opening of the chamber outlet 13 does not overlap the light source aperture 14 when viewed from the direction of the central axis L1 of the light source aperture 14. In other words, even when viewed from the direction of the central axis L3 of the chamber outlet 13, the chamber outlet 13 may be disposed so that the entire opening of the chamber outlet 13 does not overlap the light source aperture 14. In this case, a strong spiral flow can be maintained in the fluid flowing to the chamber outlet 13.

Furthermore, the inner diameter of the chamber outlet 13 is larger than that of the chamber inlet 12. By reducing the inner diameter of the chamber inlet 12, the flow rate of the fluid flowing into the sterilization chamber 60 can be increased, a spiral flow can be effectively generated. Furthermore, by increasing the inner diameter of the chamber outlet 13, the fluid can flow out while maintaining the spiral flow. Therefore, the pressure loss near the chamber outlet 13 can be reduced, and the sterilization efficiency can be improved.

Additionally, when viewed from the direction shown in FIG. 4, the ratio of the area outside the light source aperture 14 to the area of the chamber inlet 12 or the chamber outlet 13 is preferably 50% or more, and the larger the ratio, the more preferable. This facilitates generating a spiral flow. The area ratio needs to be set according to the diameter D of the sterilization chamber 60, the inner diameter di of the chamber inlet 12, and the inner diameter do of the chamber outlet 13.

### 5. Configuration of Boundary Surfaces 10Aa and 10Ba and First Seal Member 40

The boundary surface 10Aa of the first body component member 10A, the boundary surface 10Ba of the second body component member 10B and the first seal member 40 are described with reference to FIGS. 3 (a) and (b).

The boundary surface 10Aa of the first body component member 10A is formed in a ring shape. A ring-shaped recessed groove 15 is formed in the boundary surface 10Aa. This recessed groove 15 is provided to fit the ring-shaped first seal member 40 in the boundary surface 10Aa and to fix the position of the first seal member 40. The cross-sectional shape of the recessed groove 15 may be any shape such as rectangle, V-shape, and circle into which the first seal member 40 can be fitted.

The first seal member 40 is made of fluororubber or fluoroelastomer. Both materials are elastic materials that are resistant to degradation caused by ultraviolet light and have a high ultraviolet light reflectance. The first seal member 40 may be of any cross-sectional shape, although a ring shape with a circular cross section is given as an example.

The first seal member 40 is fitted into the recessed groove 15. By fitting the first seal member 40 into the recessed groove 15, the first seal member 40 can be fixed and stabilized and positioned on the boundary surface 10Aa of the first body component member 10A. In addition, by fitting the first seal member 40 into the recessed groove 15, the exposed area of the first seal member 40 can be reduced so that the first seal member 40 is not directly irradiated with ultraviolet light. Therefore, the service life of the first seal member 40 can be improved.

As shown in FIG. 3(b), in the fluid sterilization device 1, the boundary surface 10Aa of the first body component member 10A and the boundary surface 10Ba of the second body component member 10B are disposed to face each other. The sterilization chamber body 10 is disposed to be pressed by the housing 30 inside the housing 30 in the vertical direction in FIG. 3(b). The pressure causes the first seal member 40 to be in close contact with the boundary surface 10Aa of the first body component member 10A and the boundary surface 10Ba of the second body component member 10B over the entire circumferential direction. In this way, the first seal member 40 separates the sterilization chamber 60 from the outside region 70.

In this state, the boundary surface 10Aa of the first body component member 10A and the boundary surface 10Ba of the second body component member 10B are in close contact with each other or have a slight gap therebetween. In this way, the sterilization chamber 60 is formed by the semiconcave surface of the first body component member 10A and the semiconcave surface of the second body component member 10B so that the sterilization chamber body 10 is formed.

Thus, even if a fluid enters the boundary surfaces 10Aa and 10Ba of the first body component member 10A and the second body component member 10B from the sterilization chamber 60, the first seal member 40 prevents the fluid from flowing outside the first seal member 40. In other words, the first seal member 40 seals the boundary surfaces 10Aa and 10Ba of the first body component member 10A and the second body component member 10B to prevent leakage of fluid between the sterilization chamber 60 and the outside region 70.

Depending on the dimensional accuracy and surface roughness of the boundary surfaces 10Aa and 10Ba, they may contact each other in a part of the circumferential direction and be separated from each other in the remaining part of the circumferential direction. Even in these cases, the first seal member 40 is in close contact with the boundary surface 10Ba of the second body component member 10B over the entire circumference. Therefore, the same effect as above is achieved.

The first seal member 40 is made of fluororubber or fluoroelastomer. Therefore, even if the ultraviolet light from the light source unit 20 enters the boundary surfaces 10Aa and 10Ba of the first body component member 10A and the second body component member 10B, the ultraviolet light can be reflected by the first seal member 40. Therefore, leakage of ultraviolet light from the boundary surfaces 10Aa and 10Ba of the first body component member 10A and the second body component member 10B to the outside of the sterilization chamber body 10 can be prevented.

In addition, since the first seal member 40 is fitted into the recessed groove 15, it is not directly irradiated with ultraviolet light from the light source aperture 14. Therefore, the service life of the first seal member 40 can be improved.

In this embodiment, the sterilization chamber body 10 consists of two body component members, the first body component 10A and the second body component 10B. However, the sterilization chamber body 10 may consist of three or more body component members. Even In such cases, the first seal member 40 is disposed on the boundary surfaces with the body component members to prevent leakage of fluid between the sterilization chamber 60 and the outside region 70 and to prevent leakage of ultraviolet light to the outside of the sterilization chamber body 10.

In this embodiment, the recessed groove 15 is formed in the first body component member 10A. However, the recessed groove 15 may be formed in the second body component member 10B, or in both of the first body component member 10A and the second body component member 10B.

### 6. Configuration of Light Source Unit 20

The configuration of the light source unit 20 is described with reference to FIG. 5. FIG. 5 is a cross-sectional view showing the configuration of the light source unit 20. The light source unit 20 is disposed so as to block the light source aperture 14 of the sterilization chamber body 10. The light source unit 20 is disposed so that the ultraviolet light emitting side of the light source unit 20 is on the sterilization chamber 60 side. The ultraviolet light emitted from the light source unit 20 enters the sterilization chamber 60 through the light source aperture 14.

The light source unit 20 has a mounting substrate 21, a light emitting element 22, a window member 23, a light source housing 24, and a gasket 25. The light source unit 20 as a whole is, for example, disk-shaped. However, the shape of the light source unit 20 may be any shape.

The mounting substrate 21 is a substrate having a mounting surface. A wiring pattern is formed on the mounting substrate 21. A wiring 80 that supplies electric power is connected to a back surface of the mounting substrate 21.

The light emitting element 22 is an element that emits ultraviolet light. For example, the light emitting element 22 is made of a group III nitride semiconductor, and the emission wavelength is 200 nm to 280 nm. Since the emission wavelength is in the UVC range, the fluid can be efficiently sterilized. The light emitting element 22 may be mounted directly on the mounting surface of the mounting substrate 21, or a LED package may be mounted on the mounting surface of the mounting substrate 21. The LED package is a unit in which the light emitting element 22 is disposed in a housing and sealed by a glass plate or lens. Various elements (e.g., Zener diodes) necessary for driving or protecting the light emitting element 22 are mounted on the mounting surface of the mounting substrate 21.

The window member 23 is a circular glass plate disposed on the gasket 25. The window member 23 is made of quartz. Any material other than quartz may be used as long as it transmits ultraviolet light. For example, sapphire may be used. The window member 23 is not limited to a plate shape, but may be lens-shaped, for example, a TIR lens, a fly-eye lens, or a Fresnel lens.

The light source housing 24 is formed so as not to cover at least the center of the window member 23, but to cover other parts of the window member 23. The light source housing 24 is formed, for example, to continuously cover the back surface and side surfaces of the mounting substrate 21 and the side surfaces of the window member 23. The light source housing 24 may consist of a single member or a plurality of members.

The light source housing 24 is formed of a material with high heat dissipation. For example, the light source housing 24 is formed of a metal such as SUS, Al, or a resin material with high heat dissipation. Since the light source housing 24 of the light source unit 20 is in contact with fluid, the light source unit 20 can be efficiently cooled.

The gasket 25 is formed in a ring shape, disposed along a vicinity of the edge on the mounting substrate 21, and the light emitting element 22 and various elements are disposed inside the gasket 25. The height of the gasket 25 is set higher than those of the light emitting elements 22 and various elements. The gasket 25 is made of an elastic material that is resistant to ultraviolet light. The gasket 25 may be made of the same material as that of the first seal member 40.

The gasket 25 is elastically deformed and is in close contact with the mounting substrate 21, the window member 23, and the light source housing 24. Thus, the interior space enclosed by the light source housing 24, the window member 23, and the gasket 25 is sealed to prevent fluid from leaking into that interior space. Thus, it is possible to prevent fluid from entering the area where the light emitting element 22 and various elements are disposed. The gasket 25 may be in close contact with only the mounting substrate 21 and the window member 23, or may be in close contact with only the window member 23 and the light source housing 24.

Here, the outer surface of the light source unit 20 has a light-emitting surface 20A that emits ultraviolet light, an external back surface 20B disposed on the back surface of the light-emitting surface, and an outer peripheral surface 20C. The light-emitting surface 20A is located corresponding to the light source aperture 14 and is formed by the part that is not covered by the light source housing 24 of the window member 23. In other words, the light-emitting surface 20A is formed by the surface of the window member 23. The external back surface 20B is formed by the back surface of the light source housing 24. The outer peripheral surface 20C is formed by the outer circumferential surface of the light source housing 24.

### 7. Configuration of Housing 30 and Second Seal Member 50

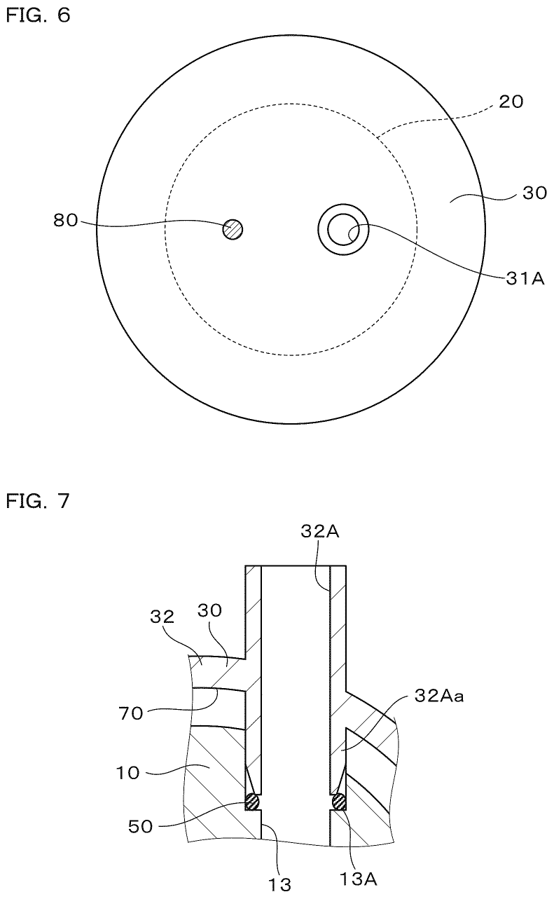

The configuration of the housing 30 and the second seal member 50 are described with reference to FIG. 1, FIG. 6, and FIG. 7. As described above, the housing 30 is formed to include the sterilization chamber body 10 and the light source unit 20. The housing 30 includes a first housing member 31 and a second housing member 32. The dividing position of the first housing member 31 and the second housing member 32 may be arbitrarily set. The housing 30 may consist of one member or three or more members. The joint between the first housing member 31 and the second housing member 32 has a seal structure not shown.

The first housing member 31 is configured to cover a part of the outer surface of the sterilization chamber body 10 and the outer surface of the light source unit 20. Accordingly, a part of the outside region 70 is formed between the inner surface of the first housing member 31, and the outer surface of the sterilization chamber body 10 and the outer surface of the light source unit 20.

The first housing member 31 has a housing inlet 31A. The housing inlet 31A is formed in a cylindrical shape, for example, a cylindrical shape or a polygonal cylindrical shape. As shown in FIG. 1 and FIG. 6, the housing inlet 31A is disposed to face the external back surface 20B of the light source unit 20. When viewed from the central axis direction of the housing inlet 31A, at least a part of the opening of the housing inlet 31A is set to face the external back surface of the light source unit 20. Thus, the fluid flowing into the outside region 70 from the housing inlet 31A can directly hit the light source unit 20, thereby improving the cooling efficiency of the light source unit 20.

In particular, the entire opening of the housing inlet 31A is preferably set to face the external back surface 20B of the light source unit 20 when viewed from the central axis direction of the housing inlet 31A. This can further improve the cooling efficiency of the light source unit 20.

Furthermore, the first housing member 31 has an opening 31B for passing the wiring 80 that connects the light source unit 20 to the outside. The opening 31B is cylindrical, for example, and one end of the cylinder is in contact with the external back surface 20B of the light source unit 20. By passing the wiring 80 through the cylinder of the opening 31B, the connection between the light source unit 20 and the wiring 80 and the wiring 80 do not come into contact with fluid.

The second housing member 32 is configured to cover the remaining part of the outer surface of the sterilization chamber body 10. A part of the outside region 70 is formed between the inner surface of the second housing member 32 and the outer surface of the sterilization chamber body 10.

A housing outlet 32A is formed in the second housing member 32. The housing outlet 32A is connected to the chamber outlet 13 of the sterilization chamber body 10. The connection structure of the chamber outlet 13 of the sterilization chamber body 10 and the housing outlet 32A of the housing 30 is described with reference to FIG. 7.

As shown in FIG. 7, the housing outlet 32A has a cylindrical portion 32Aa protruding toward the chamber outlet 13 and is connected to the chamber outlet 13 by fitting the cylindrical portion 32Aa into the chamber outlet 13. The area of the housing outlet 32A side of the chamber outlet 13 has an inner diameter larger than the inner diameter of the other areas, and the inner diameter is approximately the same as the outer diameter of the cylindrical portion 32Aa above.

This difference in the inner diameter forms a step portion 13A at the chamber outlet 13. A ring-shaped second seal member 50 is disposed in this step portion 13A. The second seal member 50 is made of the same material as that of the first seal member 40. That is, the second seal member 50 is formed of fluororubber or fluoroelastomer.

The second seal member 50 is formed in a ring shape. The second seal member 50 is disposed between the boundary surfaces of the chamber outlet 13 and the housing outlet 32A. The second seal member 50 is elastically deformed by the pressure from the housing 30 and is in close contact with both the step portion 13A of the chamber outlet 13 and the tip of the cylindrical portion 32Aa of the housing outlet 32A. With this structure, the second seal member 50 seals the boundary surfaces of the chamber outlet 13 and housing outlet 32A. Thus, the second seal member 50 prevents leakage of fluid between the chamber outlet 13 and the outside region 70 through the boundary surface between the chamber outlet 13 and the housing outlet 32A.

### 8. Water Flow Simulation

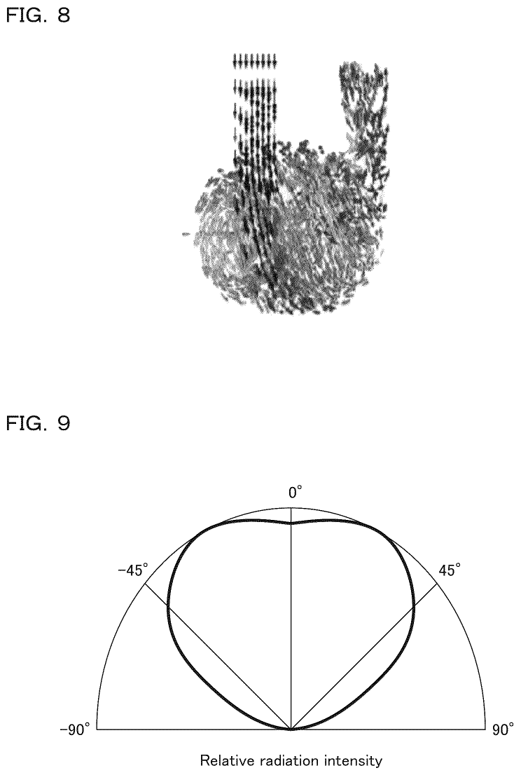

The sterilization chamber 60 in this embodiment was modeled and water flow simulations were performed. The water flow simulation results show that a spiral flow is formed in the sterilization chamber 60, as shown in FIG. 8.

When the flow rate per unit time of water circulating through the sterilization chamber 60 was 8 L/sec., the time from when the water flowed in the chamber inlet 12 to when the water flowed out from the chamber outlet 13, i.e., the retention time of water in the sterilization chamber 60, was 0.14 seconds. The sterilization performance of the water flown out was very high, and the sterilization or inactivation of the target bacteria and viruses was confirmed to be 90% or more.

In view of the above, the sterilization chamber body 10 may be configured such that when the flow rate per unit time of the fluid circulating through the sterilization chamber 60 L/sec. is 0.5 to 50 L/sec., the retention time of the fluid in the sterilization chamber 60 is 0.02 to 2 seconds. Thus, the desired cumulative irradiation amount can be ensured, and the desired sterilization performance can be ensured.

### 9. Details of Characteristics of Light Emitting Element 22.

Details of the various characteristics of the light emitting element 22 of the light source unit 20 are listed below.

9-1. Emission Efficiency at a Current Value within a Range of 300 mA to 400 mA

The light emitting element 22 is configured to have an emission efficiency (ratio of total radiation flux to input power) of 2.4% or more at a current value within a range of 300 mA to 400 mA is. This means that the emission efficiency being a function of the current value, has a minimum value of 2.4% or more at the current value within the range of 300 mA to 400 mA. Therefore, with appropriate power input, sufficient emission intensity can be obtained while suppressing the heat generation of the light emitting element 22. As a result, the sterilization performance of the fluid sterilization device can be improved. The emission efficiency is more preferably 2.42% or more, and further preferably 2.45% or more at the current value within the range of 300 mA to 400 mA

Furthermore, an emission efficiency at a current value of 350 mA is preferably 2.5% or more, more preferably, 2.55% or more, and further preferably, 2.6% or more.

The maximum value of the emission efficiency is not specified at the current value within the range of 300 mA to 400 mA, but a variation in the emission efficiency is preferably small. For example, a difference between the maximum and minimum values of the emission efficiency is preferably 0.3% or less at the current value within the range of 300 mA to 400 mA.

The forward voltage of the light emitting element 22 is, for example, 4 V to 6 V at the current value within the range of 300 mA to 400 mA. The total radiation flux of the light emitting element 22 is, for example, 10 mW to 70 mW at the current value within the range of 300 mA to 400 mA.

### 9-2. Half Width of Emission Spectrum

The light emitting element 22 is configured so that a difference between a maximum half width value and a minimum half width value in an emission spectrum is 2 nm or less at a current value within a range of 200 mA to 500 mA. Thus, a variation in the half width by the current value becomes small. Therefore, the difference in sterilization performance of the fluid sterilization device due to fluctuations in the current value becomes small, and stable sterilization performance can be obtained. More preferably, the difference between the maximum half width value and the minimum half width value is 1 nm or less, and more preferably, 0.5 nm or less at the current value within the range of 200 mA to 500 mA.

The lower limit of the difference between the maximum half width value and the minimum half width value is not specified at the current value within the range of 200 mA to 500 mA, but the smaller difference is more preferable. In terms of feasibility and manufacturing cost, the difference between the maximum half width value and the minimum half width value is preferably 0.01 nm or more at the current value within the range of 200 mA to 500 mA.

The maximum half width value is, for example, 10 nm to 15 nm at the current value within the range of 200 mA to 500 mA.

9-3. Emission Efficiency at a Current Value within a Range of 10 mA to 50 mA and 200 mA to 500 mA

The light emitting element 22 is configured such that an emission efficiency at one of current values within a range of 10 mA to 50 mA takes a maximum value among emission efficiencies at applied current values, and such that the emission efficiencies at current values in an entire range of 200 mA to 500 mA are 75% or more relative to a maximum value of the emission efficiency at a current value within a range of 200 mA to 500 mA. Here, the applied current value is within a range of 0 mA to the maximum current value that is actually expected to be used. Therefore, the decrease in emission efficiency due to an increase in the current value becomes small. Therefore, the difference in sterilization performance of the fluid sterilization device due to fluctuations in the current value becomes small, and stable sterilization performance can be obtained. The emission efficiencies are 78% or more, and more preferably 80% or more, relative to the maximum value of the emission efficiency at the current value within a range of 200 mA to 500 mA.

Furthermore, the light emitting element 22 is preferably configured such that the emission efficiencies at current values in the entire range of 200 mA to 500 mA fall within a range of 65% to 85% relative to the maximum value of the emission efficiency at the applied current values. More stable sterilization performance can be obtained. The emission efficiencies are more preferably 67% to 83%, and further preferably 70% to 80%.

A peak wavelength shift of the light emitting element 22 is preferably 1 nm or less at the current value within the range of 200 mA to 500 mA. The peak wavelength shift by the current value becomes small, and a variation in sterilization performance due to fluctuations in the current value also becomes small. Therefore, stable sterilization performance can be obtained.

### 9-4. Examples of Specific Numerical Values of Various Characteristics of Light Emitting Element 22

An example of specific numerical values of various characteristics of the light emitting element 22 is shown in Table 1 below. Table 1 summarizes the values of forward voltage VF (V), total radiation flux (mW), emission efficiency (%), peak wavelength (nm), and half width of emission spectrum (nm) at each current value IF (mA) of the light emitting element 22.

TABLE 1

| | | Total radiation | Emission | Peak | Half |
|---|---|---|---|---|---|
| | | flux | efficiency | wavelength | width |
| IF(mA) | VF(V) | (mW) | (%) | (nm) | (nm) |
| 1 | 4.27 | 0.10 | 2.39 | 287.2 | 17.5 |
| 2 | 4.34 | 0.24 | 2.80 | 286.2 | 16.6 |
| 5 | 4.44 | 0.70 | 3.16 | 285.0 | 15.8 |
| 10 | 4.52 | 1.51 | 3.34 | 284.1 | 15.2 |
| 20 | 4.63 | 3.17 | 3.43 | 283.3 | 14.4 |
| 50 | 4.79 | 8.10 | 3.38 | 282.3 | 13.7 |
| 100 | 4.95 | 15.77 | 3.19 | 281.6 | 13.2 |
| 200 | 5.16 | 29.61 | 2.87 | 281.0 | 12.8 |
| 350 | 5.40 | 47.38 | 2.51 | 280.7 | 12.7 |
| 500 | 5.58 | 62.51 | 2.24 | 280.6 | 12.7 |

### 9-5. Examples of Other Element Configurations and Characteristics

Examples of the preferable configuration and characteristics of the light emitting elements 22 are listed below.

FIG. 9 shows an example of the orientation characteristics of the light emitting element 22. The direction perpendicular to the main surface of the light emitting element 22 is set to 0°. As shown in FIG. 9, the radiation intensity has peaks in two oblique directions, a + angle and a − angle relative to 0°. The 0° direction is not a peak of the radiation intensity. The peaks are, for example, in the −50° to −15° direction and in the +15° to +50° direction. The radiation intensity does not necessarily have to be symmetrical with respect to 0°.

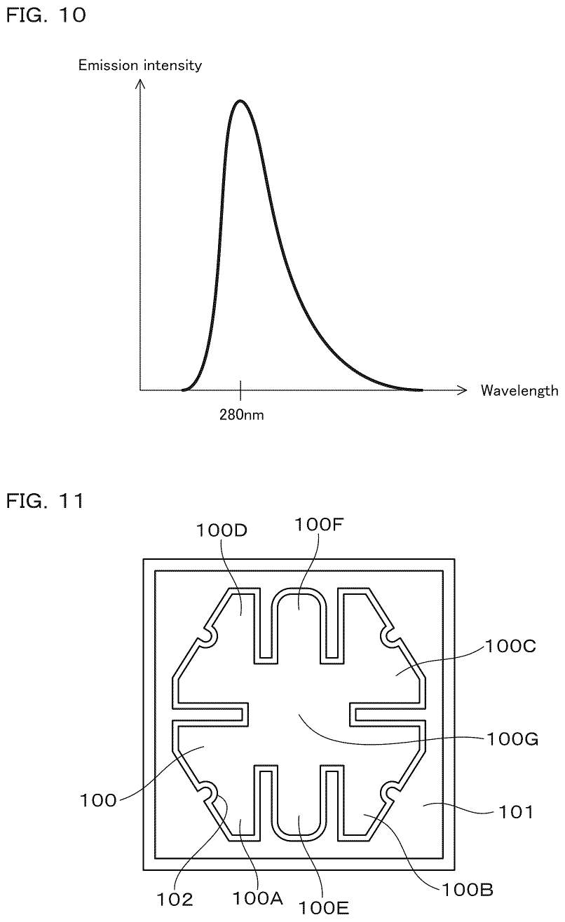

FIG. 10 shows an example of the emission spectrum of the light emitting elements 22. As shown in FIG. 10, the spectrum has a peak at around 280 nm, and the spectrum is wider at wavelengths longer than the peak wavelength than at wavelengths shorter than the peak wavelength. In other words, the half-width at half maximum is larger at wavelengths longer than the peak wavelength than at wavelengths shorter than the peak wavelength.

FIG. 11 shows an example of a pattern of pad electrode in the light emitting element 22. As shown in FIG. 11, a p-pad electrode 100 has four right triangle shaped areas 100A to 100D, two rectangle shaped areas 100E and 100F, and a central area 100G.

The areas 100A to 100D are shaped as right triangles with the non-right-angled corners of the triangles truncated by straight lines parallel to the sides forming the right angles. The areas 100A to 100D are arranged symmetrically right and left and up and down, with the right-angled corners on the central area 100G side, and the right-angled corners of the areas 100A to 100D are connected to the central portion 100G of the p-pad portion. The oblique sides of the right triangles are provided with recesses 102 recessed to the right-angled corner side.

The area 100E is disposed between the areas 100A and 100B, and one end of the area 100E is connected to the central area 100G. Other two corners of the area 100E are rounded. The area 100F is disposed between the areas 100C and 100D, one end of the area 100F is connected to the central area 100G, and extends to the side opposite to the area 100E. Other two corners of the area 100F are rounded.

An n-pad electrode 101 is formed over an almost entire area except for the p-pad electrode 100 of the rectangular element area.

The light emitting layers of the light emitting element 22 may have an MQW structure having two well layers. This makes it easier to satisfy the numerical ranges of the emission efficiency and the half width described above.

What is claimed is:

1. A fluid sterilization device comprising:
a sterilization chamber body including
a sterilization chamber for a fluid, a wall surface of which has a concave spherical shape,
a light source aperture,
a chamber inlet for inflowing the fluid into the sterilization chamber, and
a chamber outlet for outflowing the fluid from the sterilization chamber, the light source aperture, the chamber inlet, and the chamber outlet being formed so as to be open to the sterilization chamber;
a light source unit that blocks the light source aperture and is configured to emit ultraviolet light from the light source aperture into the sterilization chamber; and
a housing that is disposed so as to cover an outer surface of the sterilization chamber body, has a housing inlet for feeding the fluid formed thereon, and has an outside region, which faces the outer surface of the sterilization chamber body and an outer surface of the light source unit, formed therein, the outside region being configured to flow the fluid fed from the housing inlet to the chamber inlet, wherein
the light source unit includes a light emitting element configured to emit the ultraviolet light.

2. The fluid sterilization device according to claim 1, wherein the light emitting element is configured to have an emission efficiency of 2.4% or more at a current value within a range of 300 mA to 400 mA.

3. The fluid sterilization device according to claim 1, wherein the light emitting element is configured such that in an emission spectrum, a difference between a maximum half width value and a minimum half width value is 2 nm or less at a current value within a range of 200 mA to 500 mA.

4. The fluid sterilization device according to claim 1, wherein
the light emitting element is configured such that an emission efficiency at one of current values within a range of 10 mA to 50 mA takes a maximum value among emission efficiencies at applied current values, and
such that the emission efficiencies at current values in an entire range of 200 mA to 500 mA are 75% or more relative to a maximum value of the emission efficiency at a current value within a range of 200 mA to 500 mA.

5. The fluid sterilization device according to claim 2, wherein the light emitting element is configured to have an emission efficiency of 2.5% or more at a current value of 350 mA.

6. The fluid sterilization device according to claim 3, wherein the light emitting element is configured such that in the emission spectrum, the difference between the maximum half width value and the minimum half width value is 1 nm or less at the current value within the range of 200 mA to 500 mA.

7. The fluid sterilization device according to claim 4, wherein the light emitting element is configured such that the emission efficiencies at current values in the entire range of 200 mA to 500 mA fall within a range of 65% to 85% relative to the maximum value of the emission efficiency at the applied current values.

8. The fluid sterilization device according to claim 2, wherein the light emitting element is configured such that in an emission spectrum, a difference between a maximum half width value and a minimum half width value is 2 nm or less at a current value within the range of 200 mA to 500 mA.

9. The fluid sterilization device according to claim 4, wherein the light emitting element is configured to have an emission efficiency of 2.4% or more at a current value within a range of 300 mA to 400 mA.

10. The fluid sterilization device according to claim 4, wherein the light emitting element is configured such that in an emission spectrum, the difference between the maximum half width value and the minimum half width value is 2 nm or less at the current value within the range of 200 mA to 500 mA.

11. The fluid sterilization device according to claim 9, wherein the light emitting element is configured such that the difference between the maximum half width value and the minimum half width value is 2 nm or less at the current value within a range of 200 mA to 500 mA.

12. The fluid sterilization device according to claim 2, wherein
the light source unit comprises:
a light-emitting surface that is located corresponding to the light source aperture and is configured to emit ultraviolet light; and
an external back surface that forms part of the outer surface of the light source unit, the external back surface being an opposite side surface of the light-emitting surface with which the fluid flowing through the outside region comes into contact, and
the housing inlet is arranged such that an opening of the housing inlet faces the external back surface of the light source unit.

13. The fluid sterilization device according to claim 3, wherein
the light source unit comprises:
a light-emitting surface that is located corresponding to the light source aperture and is configured to emit ultraviolet light; and
an external back surface that forms part of the outer surface of the light source unit, the external back surface being an opposite side surface of the light-emitting surface with which the fluid flowing through the outside region comes into contact, and
the housing inlet is arranged such that an opening of the housing inlet faces the external back surface of the light source unit.

14. The fluid sterilization device according to claim 4, wherein
the light source unit comprises:
a light-emitting surface that is located corresponding to the light source aperture and is configured to emit ultraviolet light; and
an external back surface that forms part of the outer surface of the light source unit, the external back surface being an opposite side surface of the light-emitting surface with which the fluid flowing through the outside region comes into contact, and
the housing inlet is arranged such that an opening of the housing inlet faces the external back surface of the light source unit.
